(19) United States
(12) Patent Application Publication
(10) Pub. No.: US 2005/0271700 A1
(43) Pub. Date: Dec. 8, 2005

(54) Poly(ester amide) coating composition for implantable devices

(76) Inventors: Jessica R. DesNoyer, San Jose, CA (US); Syed F.A. Hossainy, Fremont, CA (US); Stephen D. Pacetti, San Jose, CA (US); Yiwen Tang, San Jose, CA (US)

Correspondence Address: Squire, Sanders & Dempsey, L.L.P., Suite 300, 1 Maritime Plaza, San Francisco, CA, 94111, US

(21) Appl. No.: 10/750,139

(22) Filed: Jun. 3, 2004

(51) Int. Cl. A61F 2/00; A61L 2/00

(52) U.S. Cl. 424/426; 427/2.24

(57) ABSTRACT

A poly(ester amide) (PEA) coating with enhanced mechanical and/or release rate for coating an implantable device, such as a drug-eluting stent, is disclosed. A method of forming the PEA coating onto a device and a method of treating a disorder, such as restenosis, are also disclosed.

### BACKGROUND OF THE INVENTION

1. Field of the Invention

This invention generally relates to a poly(ester amide) composition for coating an implantable device such as a drug-eluting stent (DES).

2. Description of the Background

Blood vessel occlusions are commonly treated by mechanically enhancing blood flow in the affected vessels, such as by employing a stent. Stents act as scaffoldings, functioning to physically hold open and, if desired, to expand the wall of the passageway. Typically stents are capable of being compressed, so that they can be inserted through small lumens via catheters, and then expanded to a larger diameter once they are at the desired location.

Stents are used not only for mechanical intervention but also as vehicles for providing biological therapy. Pharmacological therapy can be achieved by medicating the stents. Medicated stents provide for the local administration of a therapeutic substance at the diseased site. Local delivery of a therapeutic substance is a preferred method of treatment because the substance is concentrated at a specific site and thus smaller total levels of medication can be administered in comparison to systemic dosages that often produce adverse or even toxic side effects for the patient. One method of medicating a stent involves the use of a polymeric carrier coated onto the surface of the stent. A composition including a solvent, a polymer dissolved in the solvent, and a therapeutic substance dispersed in the blend is applied to the stent by immersing the stent in the composition or by spraying the composition onto the stent. The solvent is allowed to evaporate, leaving on the stent surfaces a coating of the polymer and the therapeutic substance impregnated in the polymer.

Generally, a polymer forming a coating composition for an implantable device has to be biologically benign. The polymer is preferably biocompatible and bioabsorbable. One such polymer family are the poly(ester amides). Poly(ester amides) can have excellent biocompatibility. However, a coating formed of PEA can incur mechanical failures caused by the coating's adhesive quality. More particularly, PEA has a tendency to adhere to the catheter balloon, which results in extensive balloon shear damage along the luminal stent surface post balloon expansion (FIG. 1). In addition, PEA, which has ester and amide functionalities in its backbone, is highly permeable to highly oxygenated drugs such as Everolimus. Everolimus has a macro-lactone structure with more than ten oxygenated functionalities that render the drug more hydrophilic than drugs that are less oxygenated. In comparison, olefinic polymers such as ethylene vinyl (EVAL) alcohol copolymer and copolymers based on polyvinylidene fluoride (for example, Kynar™ and Solef™) are less permeable to highly oxygenated drugs such as Everolimus. In order to achieve a proper level of residence time of an agent in a PEA stent, it would require thicker coatings to meet release rate targets.

Therefore, there is a need for a PEA coating composition that provides for a controlled release of a bioactive agent and improved mechanical properties.

The compositions and the coatings formed thereof disclosed herein address the above described problems and needs that are apparent to one having ordinary skill in the art.

### SUMMARY OF THE INVENTION

Provided herein is a method for improving the surface and mechanical properties of a coating comprising poly(ester amide) (PEA) on an implantable device. Generally, the method comprises lowering the surface energy of the PEA coating. In one aspect, the composition comprises PEA, a low surface energy, surface blooming polymer and optionally a bioactive agent. The low surface energy polymer comprises a block or component that is miscible with the PEA polymer and a surface blooming block, pendant groups or a component. The low surface energy, surface blooming polymer may have one of the following general formulae:
A-B (I),
B-A-B (II),
B [symbol] A-B)ₙ (III),
and 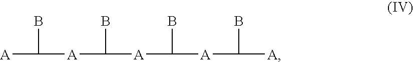
wherein A is a PEA miscible block or PEA miscible backbone, and wherein B is a surface blooming block or surface blooming pendant group. In one embodiment, A can be, for example, one of polyurethane, poly(ester-urea) urethane, polyglycol, poly(tetramethylene glycol), poly(propylene glycol), polycaprolactone, ethylene vinyl alcohol copolymer, poly(butyl methacrylate), poly(methacrylate), poly(acrylate), poly(ether-urethane), poly(ester-urethane), poly(carbonate-urethane), poly(silicone-urethane), poly(urea-urethane), poly(glycolide), poly(L-latide), poly(1-lactide-co-glycolide), poly(D,L-lactide), poly(D,L-lactide-co-glycolide), poly(D,L-lactide-co-L-lactide), poly(glycolide-co-caprolactone), poly(D,L-lactide-co-caprolactone), poly(L-lactide-co-caprolactone), poly(dioxanone), poly(trimethylene carbonate), poly(trimethylene carbonate) copolymers, poly(3-hydroxybutyrate), poly(3-hydroxyvalerate), poly(4-hydroxybutyrate), poly(3-hydroxybutyrate-co-3-hydroxyvalerate), styrene-butadiene-styrene block copolymer, styrene-butylene/ethylene-styrene block copolymer, styrene-isobutylene-styrene triblock copolymer, poly(ethylene-co-vinyl acetate), and a combination thereof, and B can be, for example, a linear or branched alkyl chain, polysilanes, polysiloxanes, poly(dimethylsiloxane), a linear or branched perfluoroalkyl chain, or a combination thereof. For example, B can be derived from any of the following materials, an organosilicone surfactant such as SILWET™ surfactants, block copolymers of alkyl chains with polyglycol chains, nonionic surfactants such as fluoro surfactants manufactured by 3M company (Fluorad™), block copolymers of polydimethylsiloxane and polycaprolactone, polyurethanes endcapped with long chain perfluoro alcohols, poly(ester-urea)urethanes endcapped with long chain perfluoro alcohols, polyurethanes endcapped with alkyl chains, polyurethanes endcapped with polydimethylsiloxane, and combinations thereof. The bioactive agent can be any active agent, for example, Everolimus, paclitaxel, docetaxel, estradiol, steroidal anti-inflammatory agents, antibiotics, anticancer agents, nitric oxide donors, super oxide dismutases, super oxide dismutases mimics, 4-amino-2,2,6,6-tetramethylpiperidine-1-oxyl (4-amino-TEMPO), ABT-578, tacrolimus, pimecrolimus, batimastat, mycophenolic acid, clobetasol, dexamethasone, rapamycin, 40-O-(3-hydroxy)propyl-rapamycin, 40-O-[2-(2-hydroxy)ethoxy]ethyl-rapamycin, or 40-O-tetrazole-rapamycin, and a combination thereof.

In another embodiment, the coating composition may comprise PEA and a low surface energy polymer additive. Low surface energy polymers are polymers that have a low polymer-air interfacial free energy. Polymer-air interface free energy can be measured in a few ways. One of the measurements is the water-air-polymer contact angle on the surface using a sessile water droplet. A polymer that has a water-air-polymer contact angle on the surface greater than 90 degrees is deemed to have a “low surface free energy” and is defined as a low surface energy polymer. Exemplary low surface energy polymers include, but are not limited to, Teflon (polytetrafluoroethylene), FEP (fluorinated ethylene-propylene or poly(tetrafluoroethylene-co-hexafluoropropene), PVDF (polyvinylidene fluoride), Silicone (polydimethylsiloxane), hydrocarbon polymers such as polyethylene; polypropylene; polystyrene and polybutadiene, and combinations thereof. In general, fluoropolymers and siloxanes or silicone polymers are the lowest surface free energy polymers.

The composition provided herein can be coated onto an implantable device. The implantable device can be any implantable device. In one embodiment, the implantable device is a DES. The implantable device can be used for the treatment of a medical condition such as atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, and tumor obstruction.

### BRIEF DESCRIPTION OF THE FIGURES

FIG. 1 is a scanning electron micrograph of PEA Benzyl Ester coated Vision stent depicting the typical type of mechanical failure observed upon deployment.

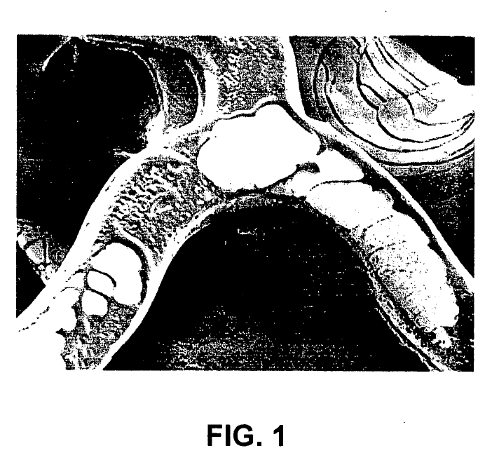

### DETAILED DESCRIPTION

### PEA Coatings with Improved Mechanical and Release Rate Properties

### Low Surface Energy Polymers

It is disclosed herein a method for improving the mechanical and release rate properties of PEA coatings by lowering the surface energy of the PEA coatings. The term poly (ester amide) is defined as a polymer having at least one ester functionality and at least one amide functionality in the backbone. The term “surface energy” refers to poly-air interface free energy. Polymer-air interface free energy can be measured in a few ways. One of the measurements is the water-air-polymer contact angle on the surface using a sessile water droplet. A polymer that has a water-air-polymer contact angle on the surface greater than 90 degrees is deemed to have a “low surface free energy” and is defined as a low surface energy polymer.

In one embodiment, the method comprises blending a PEA with one or more low surface energy polymer additives. Low surface energy polymer additives are polymers that have a low polymer-air interfacial free energy. Exemplary low surface energy polymers include, but are not limited to, Teflon (polytetrafluoroethylene), FEP (fluorinated ethylene-propylene), poly(tetrafluoroethylene-co-hexafluoropropene), PVDF (polyvinylidene fluoride), poly(fluoroalkenes), polysilanes, polysiloxanes, silicone (polydimethylsiloxane), hydrocarbon polymers such as polyethylene, polypropylene, polystyrene and polybutadiene, and combinations thereof. In general, fluoropolymers and polysiloxanes or silicone polymers are the lowest surface free energy polymers. Optionally, the method described herein may comprise blending a bioactive agent into PEA and the low surface energy polymer additive.

In another embodiment, the method described herein comprises blending a PEA with one or more low surface energy, surface blooming polymer. The low surface energy, surface blooming polymer may comprise two components, one being miscible with the PEA polymer in the coating composition, and the other is a hydrophobic blooming component. In the PEA coating, the surface is enriched with the hydrophobic blooming component. This would reduce or prevent the interaction between the PEA polymer and the catheter balloon, thereby reducing potential mechanical failures of a PEA coating on an implantable device. Additionally, the hydrophobic, blooming component of the coating would create a hydrophobic barrier at the coating surface, thereby retarding drug release from the PEA matrix. As a result, thinner coatings can be used to obtain the same release rate control of a thicker coating of PEA polymer matrix. Further, the hydrophobic coating would further reduce the interaction between water and the PEA matrix so as to reduce the degradation rate of the PEA polymer. It is noteworthy that rapid degradation of PEA may cause or promote inflammation. A reduced rate of degradation of the PEA polymer can be desirable.

As used herein, the term “hydrophobic component” refers to a component having a hydrophobicity greater than that of PEA. Generally, hydrophobicity of a polymer can be gauged using the Hildebrand solubility parameter δ. The term “Hildebrand solubility parameter” refers to a parameter indicating the cohesive energy density of a substance. The δ parameter is determined as follows:
δ=(ΔE/V)^{1/2}
where δ is the solubility parameter, (cal/cm³)^{1/2};
ΔE is the energy of vaporization, cal/mole; and
V is the molar volume, cm³/mole.

If a blend of a hydrophobic and hydrophilic polymer(s) is used, whichever polymer in the blend has lower δ value compared to the δ value of the other polymer in the blend is designated as a hydrophobic polymer, and the polymer with higher δ value is designated as a hydrophilic polymer. If more than two polymers are used in the blend, then each can be ranked in order of its δ value. For the practice of the present invention, the value of δ of a particular polymer is inconsequential for classifying a polymer as hydrophobic or hydrophilic. The component having a δ value lower than that of PEA is designated as hydrophobic.

The low surface energy polymer comprises a block or component that is miscible with the PEA polymer and a surface blooming block, pendant groups or a component. The low surface energy, surface blooming polymer may have one of the following general formulae:
A-B (I),
B-A-B (II),
B- [symbol] A-B)ₙ (III),
and 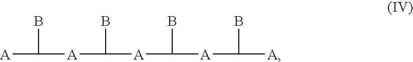
wherein A is a PEA miscible block or PEA miscible backbone, and wherein B is a surface blooming block or surface blooming pendant group.

In one embodiment, A can be, for example, one of polyurethane, poly(ester-urea) urethane, polyglycol, poly(tetramethylene glycol), poly(propylene glycol), polycaprolactone, ethylene vinyl alcohol copolymer, poly(butyl methacrylate), poly(methacrylate), poly(acrylate), and a combination thereof. B can be, for example, a linear or branched alkyl chain, polysilanes, polysiloxanes, poly(dimethylsiloxane), a linear or branched perfluoroalkyl chain, poly(ether-urethane), poly(ester-urethane), poly(carbonate-urethane), poly(silicone-urethane), poly(urea-urethane), poly(glycolide), poly(L-latide), poly(1-lactide-co-glycolide), poly(D,L-lactide), poly(D,L-lactide-co-glycolide), poly(D,L-lactide-co-L-lactide), poly(glycolide-co-caprolactone), poly(D,L-lactide-co-caprolactone), poly(L-lactide-co-caprolactone), poly(dioxanone), poly(trimethylene carbonate), poly(trimethylene carbonate) copolymers, poly(3-hydroxybutyrate), poly(3-hydroxyvalerate), poly(4-hydroxybutyrate), poly(3-hydroxybutyrate-co-3-hydroxyvalerate), styrene-butadiene-styrene block copolymer, styrene-butylene/ethylene-styrene block copolymer, styrene-isobutylene-styrene triblock copolymer, poly(ethylene-co-vinyl acetate), and a combination thereof; and B can be, for example, a linear or branched alkyl chain, polysilanes, polysiloxanes, poly(dimethylsiloxane), a linear or branched perfluoroalkyl chain, and a combination thereof. For example, B can be any of the following materials, an organosilicone surfactant such as SILWET™ surfactants, block copolymers of alkyl chains with polyglycol chains, nonionic surfactants such as fluoro surfactants manufactured by 3M company (Fluorad™), block copolymers of polydimethylsiloxane and polycaprolactone, polyurethanes endcapped with long chain perfluoro alcohols, poly(ester-urea)urethanes endcapped with long chain perfluoro alcohols, polyurethanes endcapped with alkyl chains, polyurethanes endcapped with polydimethylsiloxane, and combinations thereof.

### Bioactive Agent

The PEA coating with enhanced mechanical and release rate properties described herein may optionally include one or more bioactive agents. The bioactive agent can be any agent which is biologically active, for example, a therapeutic, prophylactic, or diagnostic agent. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Compounds with a wide range of molecular weight, for example, between about 100 and about 500,000 grams or more per mole or between about 100 and about 500,000 grams or more per mole, can be encapsulated. Some other examples of suitable materials include proteins such as antibodies, receptor ligands, and enzymes, peptides such as adhesion peptides, and saccharides and polysaccharides. Some further examples of materials which can be included in the PEA coating include blood clotting factors, inhibitors or clot dissolving agents such as streptokinase and tissue plasminogen activator, antigens for immunization, hormones and growth factors, polysaccharides such as heparin, oligonucleotides such as antisense oligonucleotides and ribozymes and retroviral vectors for use in gene therapy. Representative diagnostic agents are agents detectable by x-ray, fluorescence, magnetic resonance imaging, radioactivity, ultrasound, computer tomagraphy (CT) and positron emission tomagraphy (PET).

In the case of controlled release, a wide range of different bioactive agents can be incorporated into a controlled release device. These include hydrophobic, hydrophilic, and high molecular weight macromolecules such as proteins. The pharmacological compound can be incorporated into polymeric coating in a percent loading of between 0.01% and 70% by weight, more preferably between 5% and 50% by weight.

In one embodiment, the bioactive agent can be for inhibiting the activity of vascular smooth muscle cells. More specifically, the bioactive agent can be aimed at inhibiting abnormal or inappropriate migration and/or proliferation of smooth muscle cells for the inhibition of restenosis. The bioactive agent can also include any substance capable of exerting a therapeutic or prophylactic effect in the practice of the present invention. For example, the bioactive agent can be for enhancing wound healing in a vascular site or improving the structural and elastic properties of the vascular site. Examples of active agents include antiproliferative substances such as actinomycin D, or derivatives and analogs thereof (manufactured by Sigma-Aldrich 1001 West Saint Paul Avenue, Milwaukee, Wis. 53233; or COSMEGEN available from Merck). Synonyms of actinomycin D include dactinomycin, actinomycin IV, actinomycin I₁, actinomycin X₁, and actinomycin C₁. The bioactive agent can also fall under the genus of antineoplastic, anti-inflammatory, antiplatelet, anticoagulant, antifibrin, antithrombin, antimitotic, antibiotic, antiallergic and antioxidant substances. Examples of such antineoplastics and/or antimitotics include paclitaxel (e.g. TAXOL® by Bristol-Myers Squibb Co., Stamford, Conn.), docetaxel (e.g. Taxotere®, from Aventis S. A., Frankfurt, Germany) methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax ä (Biogen, Inc., Cambridge, Mass.). Examples of such cytostatic or antiproliferative agents include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.); calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor®. from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimics, 4-amino-2,2,6,6-tetramethylpiperidine-1-oxyl (4-amino-TEMPO), sirolimus (rapamycin) and sirolimus derivatives, docetaxel, paclitaxel and paclitaxel derivatives, estradiol, steroidal anti-inflammatory agents, antibiotics, anticancer agents, dietary supplements such as various vitamins, and a combination thereof. An example of an antiallergic agent is permirolast potassium. Other therapeutic substances or agents which may be appropriate include alpha-interferon, genetically engineered epithelial cells, Everolimus, steroidal anti-inflammatory agents, antibiotics, anticancer agents, nitric oxide donors, super oxide dismutases, super oxide dismutases mimics, 4-amino-2,2,6,6-tetramethylpiperidine-1-oxyl (4-amino-TEMPO), ABT-578, tacrolimus, pimecrolimus, batimastat, mycophenolic acid, clobetasol, dexamethasone, rapamycin, 40-O-(3-hydroxy)propyl-rapamycin, 40-O-[2-(2-hydroxy)ethoxy]ethyl-rapamycin, or 40-O-tetrazole-rapamycin, antiproliferative agents, non-steroidal anti-inflammatory agents, immunosuppressive agents, and antimigratory agents, and a combination thereof. The foregoing substances are listed by way of example and are not meant to be limiting. Other active agents which are currently available or that may be developed in the future are equally applicable.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent required to inhibit the desired cellular activity of the vascular region can depend upon factors such as the particular circumstances of the patient; the nature of the trauma; the nature of the therapy desired; the time over which the ingredient administered resides at the vascular site; and if other active agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

### Methods of Forming PEA Coatings

The hydrophobic barrier on the surface of a PEA coating can be generated by coating onto an implantable device such as a DES a composition comprising a PEA polymer, spray solvent, a low surface energy polymer, and optionally one or more bioactive agents. The composition can be in the form of a homogeneous solution, an emulsion of two liquid phases, or a dispersion or latex. The dispersed phase of the dispersion or latex would consist of nano or microparticles of the PEA polymer, low surface energy polymer, and optionally, a bioactive agent. The microparticles can have a size, for example, between 1 nanometer and 100 microns, preferably between 10 nanometers and 10 microns, more preferably between 10 nanometers and 1 micron. During the spray coating process, the low surface energy polymer will reside substantially at the air/liquid interface of the spray droplet. As the solvent evaporates, the coating surface becomes enriched with the low surface energy polymer, and the PEA component is pushed into the coating interior, thus preventing an interaction between PEA and the catheter balloon.

As used herein, the term “solvent” is defined as a liquid substance or composition that is compatible with the polymer and is capable of dissolving or suspending the polymer, a material providing biological benefit, and optionally the bioactive agent at the concentration desired in the composition. The term “a material providing biological benefit” refers to any material or polymer that can increase the biocompatibility of the PEA coating. Representative materials providing biological benefit include, for example, poly(ethylene glycol), poly(alkylene oxide) such as poly(ethylene oxide), PolyActive™, and hyaluronic acid and a salt thereof. Representative examples of solvents include chloroform, acetone, water (such as buffered saline), dimethylsulfoxide (DMSO), propylene glycol methyl ether (PM,) iso-propyl alcohol (IPA), n-propyl alcohol, methanol, ethanol, tetrahydrofuran (THF), dimethylformamide (DMF), dimethyl acetamide (DMAC), benzene, toluene, xylene, hexane, cyclohexane, heptane, octane, nonane, decane, decalin, ethyl acetate, butyl acetate, isobutyl acetate, isopropyl acetate, butanol, diacetone alcohol, benzyl alcohol, 2-butanone, cyclohexanone, dioxane, methylene chloride, carbon tetrachloride, tetrachloroethylene, tetrachloro ethane, chlorobenzene, 1,1,1-trichloroethane, formamide, hexafluoroisopropanol, 1,1,1-trifluoroethanol, and hexamethyl phosphoramide and a combination thereof.

The PEA coating described herein can be formed as a single layer of coating on an implantable device, on top of a polymer-free drug layer, on top of a polymer reservoir layer containing a drug, or in conjunction with or blend with other polymers. Other polymers that could be used in combination with PEA include, but not limited to, polylakanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalknaote) such as poly(4-hydroxybutyrate), poly(4-hydroxyvalerate), poly(4-hydroxyhexanote), poly(4-hydroxyheptanoate), poly(4-hydroxyoctanoate) and copolymers comprising any of the 3-hydroxyalkanoate or 4-hydroxyalkanoate monomers described herein or blends thereof, poly polyesters, poly(D,L-lactide), poly(L-lactide), polyglycolide, poly(lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(phosphoesters), poly(phosphazenes), poly(amino acids), polysaccharides, collagen, chitosan, alginate, and a combination thereof.

### Implantable Devices

The methods and the PEA coatings described herein are applicable to PEA coatings on any implantable device. As used herein, an implantable device may be any suitable medical substrate that can be implanted in a human or veterinary patient. A preferred implantable device is a DES. Examples of stents include self-expandable stents, balloon-expandable stents, and stent-grafts. Other exemplary implantable devices include grafts (e.g., aortic grafts), artificial heart valves, cerebrospinal fluid shunts, pacemaker electrodes, and endocardial leads (e.g., FINELINE and ENDOTAK, available from Guidant Corporation, Santa Clara, Calif.). The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention.

### Method of Use

In accordance with embodiments of the invention, a coating of the various described embodiments can be formed on an implantable device or prosthesis, e.g., a stent. For coatings including one or more active agents, the agent will be retained on the medical device such as a stent during delivery and expansion of the device, and released at a desired rate and for a predetermined duration of time at the site of implantation. Preferably, the medical device is a stent. A stent having the above-described coating is useful for a variety of medical procedures, including, by way of example, treatment of obstructions caused by tumors in bile ducts, esophagus, trachea/bronchi and other biological passageways. A stent having the above-described coating is particularly useful for treating occluded regions of blood vessels caused by atherosclerosis, abnormal or inappropriate migration and proliferation of smooth muscle cells, thrombosis, restenosis and the treatment of vulnerable plaque. Stents may be placed in a wide array of blood vessels, both arteries and veins. Representative examples of sites include the iliac, renal, and coronary arteries.

For implantation of a stent, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter which allows a stent in its collapsed configuration to be inserted into the passageway. The delivery catheter is inserted either percutaneously or by surgery into the femoral artery, brachial artery, femoral vein, or brachial vein, and advanced into the appropriate blood vessel by steering the catheter through the vascular system under fluoroscopic guidance. A stent having the above-described coating may then be expanded at the desired area of treatment. A post-insertion angiogram may also be utilized to confirm appropriate positioning.

The implantable device comprising a coating described herein can be used to treat an animal having a condition or disorder that requires a treatment. Such an animal can be treated by, for example, implanting a device described herein in the animal. Preferably, the animal is a human being. Exemplary disorders or conditions that can be treated by the method disclosed herein include, but not limited to, atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, and tumor obstruction.

### EXAMPLES

The embodiments of the present invention will be illustrated by the following set forth examples. All parameters and data are not to be construed to unduly limit the scope of the embodiments of the invention.

### Example 1

One useful surface blooming composition would be a B-A-B triblock copolymer wherein B is a mono-functional fluorinated alcohol component known as BA-L (available from Du Pont de Nemours, Wilmington, Del.), and A is a hydroxy terminated poly(caprolactone) of molecular weight 1000 known as CAPA 210 (available from Solvay Interox, Houston, Tex., USA). Synthesis of the triblock is accomplished by using 1,6-hexanediisocyanate (HDI,) and an appropriate catalyst such as dibutyltin dilaurate, in a solvent such as dimethylacetamide using what is essentially standard urethane chemistry. In this synthesis, the monofunctional fluoroalcohol is first reacted with two equivalents of HDI. Addition of the hydroxy-terminated polycaprolactone to the now isocyanate functionalized fluorocompounds produces the triblock copolymer. This surface blooming compound can be used in a PEA composition for coating a drug eluting stent.

A first composition can be prepared by mixing the following components:
(a) about 2.0 mass % of a poly(ester amide);
(b) about 1.0 mass % of Everolimus; and
(c) the balance, anhydrous ethanol.

The first composition can be applied onto the surface of a bare 12 mm VISION™ stent by spraying and dried to form a drug reservoir layer. An EFD spray head can be used, having a 0.014 inch round nozzle tip and a 0.028 inch round air cap with a feed pressure of about 0.2 atm (3 psi) and an atomization pressure of between about 1 atm and 1.3 atm (15 to 20 psi). The total amount of solids of the reservoir layer can be about 167 micrograms (μg). After spraying, the stents can be baked at about 50° C. for about one hour. “Solids” means the amount of dry residue deposited on the stent after all volatile organic compounds (e.g. the solvent) have been removed.

A second composition can be prepared by mixing the following components:
(a) about 2 mass % of poly(ester amide);
(b) about 0.05% of the surface blooming composition;
(c) the balance, a 80/20 blend of anhydrous ethanol and dimethylacetamide.

The second composition can be applied onto the dried reservoir layer to form a topcoat layer with non-adhesive properties, using the same spraying technique and equipment used for the primer layer. Solvent can be removed by baking at about 50° C. for about one hour. The total amount of solids of the topcoat layer can be about 100 μg.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A method for forming a poly(ester amide) (PEA) coating with enhanced mechanical and release rate properties, comprising:
applying to an implantable device a solution or suspension of a composition comprising PEA and a low surface energy, surface blooming polymer, and
forming a coating on the implantable device comprising PEA and the low surface energy, surface blooming polymer.

2. The method of claim 1 wherein the low surface energy, surface blooming polymer is selected from the group consisting of a block copolymer comprising a block miscible with the PEA and a hydrophobic block, a polymer comprising a backbone miscible with PEA and hydrophobic pendant groups, and a combination thereof,
wherein the hydrophobic block has a 6 value below than that of PEA.

3. The method of claim 1 wherein the low surface energy polymer is selected from the group consisting of formulae I-IV of the following structure:
A-B (I),
B-A-B (II),
B [symbol] A-B)ₙ (III),
and 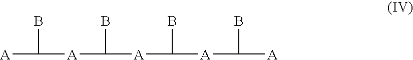
wherein A is a PEA miscible block or PEA miscible backbone, and
wherein B is selected from the group consisting of a surface blooming block and a surface blooming pendant group.

4. The method of claim 3 wherein A is selected from the group consisting of polyurethane, poly(ester-urea) urethane, polyglycol, poly(tetramethylene glycol), poly(propylene glycol), polycaprolactone, ethylene vinyl alcohol copolymer, poly(butyl methacrylate), poly(methacrylate), poly(acrylate), poly(ether-urethane), poly(ester-urethane), poly(carbonate-urethane), poly(silicone-urethane), poly(urea-urethane), poly(glycolide), poly(L-latide), poly(1-lactide-co-glycolide), poly(D,L-lactide), poly(D,L-lactide-co-glycolide), poly(D,L-lactide-co-L-lactide), poly(glycolide-co-caprolactone), poly(D,L-lactide-co-caprolactone), poly(L-lactide-co-caprolactone), poly(dioxanone), poly(trimethylene carbonate), poly(trimethylene carbonate) copolymers, poly(3-hydroxybutyrate), poly(3-hydroxyvalerate), poly(4-hydroxybutyrate), poly(3-hydroxybutyrate-co-3-hydroxyvalerate), styrene-butadiene-styrene block copolymer, styrene-butylene/ethylene-styrene block copolymer, styrene-isobutylene-styrene triblock copolymer, poly(ethylene-co-vinyl acetate), and a combination thereof; and
wherein B is selected from the group consisting of a linear or branched alkyl chain, polysilanes, polysiloxanes, poly(dimethylsiloxane), a linear or branched perfluoro chain, and a combination thereof.

5. The method of claim 1 wherein the low surface energy polymer is selected from the group consisting of organosilicone surfactants, block copolymers of alkyl chains with polyglycol chains, fluoro surfactants, block copolymers of polydimethylsiloxane and polycaprolactone, polyurethanes end-capped with long chain perfluoro alcohols, poly(ester-urea)urethanes end-capped with long chain perfluoroalcohols, polyurethanes end-capped with alkyl chains, polyurethanes end-capped with polydimethylsiloxane, copolymers of polycaprolactone and fluoroalcohols, and combinations thereof.

6. The method of any of claims 1-5 wherein the composition further comprises a bioactive agent.

7. The method of claim 6 wherein the bioactive agent is selected from the group consisting of Everolimus, paclitaxel, docetaxel, estradiol, steroidal anti-inflammatory agents, antibiotics, anticancer agents, nitric oxide donors, super oxide dismutases, super oxide dismutases mimics, 4-amino-2,2,6,6-tetramethylpiperidine-1-oxyl (4-amino-TEMPO), ABT-578, tacrolimus, pimecrolimus, batimastat, mycophenolic acid, clobetasol, dexamethasone, rapamycin, 40-O-(3-hydroxy)propyl-rapamycin, 40-O-[2-(2-hydroxy)ethoxy]ethyl-rapamycin, or 40-O-tetrazole-rapamycin, antiproliferative agents, non-steroidal anti-inflammatory agents, immunosuppressive agents, antimigratory agents, and a combination thereof.

8. A method for forming a poly(ester amide) (PEA) coating with enhanced mechanical and release rate properties, comprising:
applying to an implantable device a solution or suspension of a composition comprising PEA and at least one low surface energy polymer additive, and
forming a coating on the implantable device comprising PEA and the at least one low surface energy polymer additive.

9. The method of claim 8 wherein the at least one low surface energy polymer additive is selected from the group consisting of Teflon (poly(tetrafluoroethylene), FEP (fluorinated ethylene-propylene), poly(tetrafluoroethylene-co-hexafluoropropene), PVDF (polyvinylidene fluoride), poly(fluoroalkenes), polysilanes, polysiloxanes, silicone (polydimethylsiloxane), hydrocarbon polymers, polyethylene, polypropylene, polystyrene, polybutadiene and combinations thereof.

10. The method of claims 8 or 9 wherein the composition further comprises a bioactive agent.

11. The method of claim 10 wherein the bioactive agent is selected from the group consisting of Everolimus, paclitaxel, docetaxel, estradiol, steroidal anti-inflammatory agents, antibiotics, anticancer agents, nitric oxide donors, super oxide dismutases, super oxide dismutases mimics, 4-amino-2,2,6,6-tetramethylpiperidine-1-oxyl (4-amino-TEMPO), ABT-578, tacrolimus, pimecrolimus, batimastat, mycophenolic acid, clobetasol, dexamethasone, rapamycin, 40-O-(3-hydroxy)propyl-rapamycin, 40-O-[2-(2-hydroxy)ethoxy]ethyl-rapamycin, or 40-O-tetrazole-rapamycin, antiproliferative agents, non-steroidal anti-inflammatory agents, immunosuppressive agents, antimigratory agents, and a combination thereof.

12. A coating composition for coating an implantable device comprising poly(ester amide) (PEA) and a low surface energy, surface blooming polymer.

13. The composition of claim 13 wherein the low surface energy, surface blooming polymer is selected from the group consisting of a block copolymer comprising a block miscible with the PEA and a hydrophobic block, a polymer comprising a backbone miscible with PEA and hydrophobic pendant groups, and a combination thereof,
wherein the hydrophobic block has a 6 value below than that of PEA.

14. The composition of claim 12 wherein the low surface energy, surface blooming polymer is selected from the group consisting of formulae I-IV of the following structure:
A-B (I),
B-A-B (II),
B [symbol] A-B)ₙ (III),
and 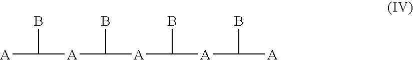
wherein A is a PEA miscible block or PEA miscible backbone, and
wherein B is selected from the group consisting of a surface blooming block and a surface blooming pendant group.

15. The composition of claim 14 wherein A is selected from the group consisting of polyurethane, poly(ester-urea) urethane, polyglycol, poly(tetramethylene glycol), poly(propylene glycol), polycaprolactone, ethylene vinyl alcohol copolymer, poly(butyl methacrylate), poly(methacrylate), poly(acrylate), and a combination thereof; and
wherein B is selected from the group consisting of a linear or branched alkyl chain, polysilanes, polysiloxanes, poly(dimethylsiloxane), a linear or branched perfluoro chain, and a combination thereof.

16. The composition of claim 15 wherein the low surface energy, surface blooming polymer is selected from the group consisting of organosilicone surfactants, block copolymers of alkyl chains with polyglycol chains, fluoro surfactants, block copolymers of polydimethylsiloxane and polycaprolactone, polyurethanes endcapped with long chain perfluoro alcohols, poly(ester-urea)urethanes endcapped with long chain perfluoro alcohols, polyurethanes endcapped with alkyl chains, polyurethanes endcapped with polydimethylsiloxane, and combinations thereof.

17. The composition of any of claims 12-16 further comprising a bioactive agent.

18. The composition of claim 17 wherein the bioactive agent is selected from the group consisting of Everolimus, paclitaxel, docetaxel, estradiol, steroidal anti-inflammatory agents, antibiotics, anticancer agents, nitric oxide donors, super oxide dismutases, super oxide dismutases mimics, 4-amino-2,2,6,6-tetramethylpiperidine-1-oxyl (4-amino-TEMPO), ABT-578, tacrolimus, pimecrolimus, batimastat, mycophenolic acid, clobetasol, dexamethasone, rapamycin, 40-O-(3-hydroxy)propyl-rapamycin, 40-O-[2-(2-hydroxy)ethoxy]ethyl-rapamycin, or 40-O-tetrazole-rapamycin, antiproliferative agents, non-steroidal anti-inflammatory agents, immunosuppressive agents, antimigratory agents, and a combination thereof.

19. A coating composition for coating an implantable device comprising poly(ester amide) (PEA) and at least one low surface energy polymer additive.

20. The composition of claim 19 wherein the at least one low surface energy polymer additive is selected from the group consisting of Teflon (poly(tetrafluoroethylene), FEP (fluorinated ethylene-propylene), poly(tetrafluoroethylene-co-hexafluoropropene), PVDF (polyvinylidene fluoride), poly(fluoroalkenes), polysilanes, polysiloxanes, silicone (polydimethylsiloxane), hydrocarbon polymers, polyethylene, polypropylene, polystyrene, polybutadiene and combinations thereof.

21. The composition of claims 19 or 20 further comprising a bioactive agent.

22. The composition of claim 21 wherein the bioactive agent is selected from the group consisting of Everolimus, paclitaxel, docetaxel, estradiol, steroidal anti-inflammatory agents, antibiotics, anticancer agents, nitric oxide donors, super oxide dismutases, super oxide dismutases mimics, 4-amino-2,2,6,6-tetramethylpiperidine-1-oxyl (4-amino-TEMPO), ABT-578, tacrolimus, pimecrolimus, batimastat, mycophenolic acid, clobetasol, dexamethasone, rapamycin, 40-O-(3-hydroxy)propyl-rapamycin, 40-O-[2-(2-hydroxy)ethoxy]ethyl-rapamycin, or 40-O-tetrazole-rapamycin, antiproliferative agents, non-steroidal anti-inflammatory agents, immunosuppressive agents, antimigratory agents, and a combination thereof.

23. An implantable device comprising a coating which comprises a poly(ester amide) (PEA) and a low surface energy, surface blooming polymer.

24. The implantable device of claim 23 wherein the low surface energy, surface blooming polymer is selected from the group consisting of a block copolymer comprising a block miscible with the PEA and a hydrophobic block, a polymer comprising a backbone miscible with PEA and hydrophobic pendant groups, and a combination thereof,
wherein the hydrophobic block has a 6 value below than that of PEA.

25. The implantable device of claim 24 wherein the low surface energy, surface blooming polymer is selected from the group consisting of formulae I-IV of the following structure:
A-B (I),
B-A-B (II),
B [symbol] A-B)ₙ (III),
and 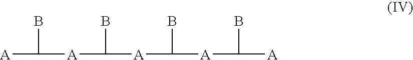
wherein A is a PEA miscible block or PEA miscible backbone, and
wherein B is selected from the group consisting of a surface blooming block and a surface blooming pendant group.

26. The implantable device of claim 25 wherein A is selected from the group consisting of polyurethane, poly(ester-urea) urethane, polyglycol, poly(tetramethylene glycol), poly(propylene glycol), polycaprolactone, ethylene vinyl alcohol copolymer, poly(butyl methacrylate), poly(methacrylate), poly(acrylate), and a combination thereof; and
wherein B is selected from the group consisting of a linear or branched alkyl chain, polysilanes, polysiloxanes, poly(dimethylsiloxane), a linear or branched perfluoro chain, and a combination thereof.

27. The implantable device of claim 26 wherein the low surface energy, surface blooming polymer is selected from the group consisting of organosilicone surfactants, block copolymers of alkyl chains with polyglycol chains, fluoro surfactants, block copolymers of polydimethylsiloxane and polycaprolactone, polyurethanes endcapped with long chain perfluoro alcohols, poly(ester-urea)urethanes endcapped with long chain perfluoro alcohols, polyurethanes endcapped with alkyl chains, polyurethanes endcapped with polydimethylsiloxane, and combinations thereof.

28. The implantable device of any of claims 23-27 further comprising a bioactive agent.

29. The implantable device of claim 28 wherein the bioactive agent is selected from the group consisting of Everolimus, paclitaxel, docetaxel, estradiol, steroidal anti-inflammatory agents, antibiotics, anticancer agents, nitric oxide donors, super oxide dismutases, super oxide dismutases mimics, 4-amino-2,2,6,6-tetramethylpiperidine-1-oxyl (4-amino-TEMPO), ABT-578, tacrolimus, pimecrolimus, batimastat, mycophenolic acid, clobetasol, dexamethasone, rapamycin, 40-O-(3-hydroxy)propyl-rapamycin, 40-O-[2-(2-hydroxy)ethoxy]ethyl-rapamycin, or 40-O-tetrazole-rapamycin, antiproliferative agents, non-steroidal anti-inflammatory agents, immunosuppressive agents, antimigratory agents, and a combination thereof.

30. An implantable device comprising a coating which comprises poly(ester amide) (PEA) and at least one low surface energy polymer additive.

31. The implantable device of claim 30 wherein the at least one low surface energy polymer additive is selected from the group consisting of Teflon (poly(tetrafluoroethylene), FEP (fluorinated ethylene-propylene), poly(tetrafluoroethylene-co-hexafluoropropene), PVDF (polyvinylidene fluoride), poly(fluoroalkenes), polysilanes, polysiloxanes, silicone (polydimethylsiloxane), hydrocarbon polymers, polyethylene, polypropylene, polystyrene, polybutadiene and combinations thereof.

32. The implantable device of claims 30 or 31 further comprising a bioactive agent.

33. The implantable device of claim 32 wherein the bioactive agent is selected from the group consisting of Everolimus, paclitaxel, docetaxel, estradiol, steroidal anti-inflammatory agents, antibiotics, anticancer agents, nitric oxide donors, super oxide dismutases, super oxide dismutases mimics, 4-amino-2,2,6,6-tetramethylpiperidine-1-oxyl (4-amino-TEMPO), ABT-578, tacrolimus, pimecrolimus, batimastat, mycophenolic acid, clobetasol, dexamethasone, rapamycin, 40-O-(3-hydroxy)propyl-rapamycin, 40-O-[2-(2-hydroxy)ethoxy]ethyl-rapamycin, or 40-O-tetrazole-rapamycin, antiproliferative agents, non-steroidal anti-inflammatory agents, immunosuppressive agents, antimigratory agents, and a combination thereof.

34. The implantable device of claim 23 which is a stent.

35. The implantable device of claim 24 which is a stent.

36. The implantable device of claim 25 which is a stent.

37. The implantable device of claim 26 which is a stent.

38. The implantable device of claim 27 which is a stent.

39. The implantable device of claim 30 which is a stent.

40. The implantable device of claim 31 which is a stent.

41. The implantable device of claim 28 which is a drug-eluting stent.

42. The implantable device of claim 29 which is a drug-eluting stent.

43. The implantable device of claim 32 which is a drug-eluting stent.

44. The implantable device of claim 33 which is a drug-eluting stent.

45. A method of treating a disorder in a human being by implanting in the human being a stent as defined in claim 34,
wherein the disorder is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

46. A method of treating a disorder in a human being by implanting in the human being a stent as defined in claim 35,
wherein the disorder is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

47. A method of treating a disorder in a human being by implanting in the human being a stent as defined in claim 36,
wherein the disorder is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

48. A method of treating a disorder in a human being by implanting in the human being a stent as defined in claim 37,
wherein the disorder is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

49. A method of treating a disorder in a human being by implanting in the human being a stent as defined in claim 38,
wherein the disorder is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

50. A method of treating a disorder in a human being by implanting in the human being a stent as defined in claim 39,
wherein the disorder is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

51. A method of treating a disorder in a human being by implanting in the human being a stent as defined in claim 42,
wherein the disorder is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

52. A method of treating a disorder in a human being by implanting in the human being a stent as defined in claim 44,
wherein the disorder is selected from the group consisting of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.
